(19) United States
(12) Patent Application Publication
(10) Pub. No.: US 2019/0315672 A1
(43) Pub. Date: Oct. 17, 2019

(54) PRODUCTION OF ACETIC ACID AND HYDROGEN IN AN AQUEOUS MEDIUM FROM ETHANOL AND ACETALDEHYDE VIA AN ORGANIC/INORGANIC CATALYST

(71) Applicant: SABIC Global Technologies B.V., Bergen op Zoom (NL)

(72) Inventors: Balamurugan Vidjayacoumar, Thuwal (SA); Khalid Albahily, Thuwal (SA); Sandro Gambarotta, Ottawa (CA); Virginie Peneau, Ottawa (CA); Nicholas P. Alderman, Ottawa (CA)

(21) Appl. No.: 16/301,587

(22) PCT Filed: May 15, 2017
(86) PCT No.: PCT/IB2017/052862

§ 371 Date: Nov. 14, 2018

Related U.S. Application Data
(60) Provisional application No. 62/343,396, filed on May 31, 2016.

(51) Int. Cl. C07C 51/16; B01J 31/22; C01B 3/32

(52) U.S. Cl. CPC C07C 51/16; B01J 31/2295; C01B 3/326; B01J 2231/763; C01B 2203/1064; C01B 2203/0227; C01B 2203/1229; C01B 2203/1211; B01J 2531/821

(57) ABSTRACT

Disclosed are methods and systems of producing acetic acid and hydrogen from a two carbon (C₂) alcohol source, the method comprising (a) obtaining a homogeneous aqueous solution comprising a C₂ alcohol source and an organoruthenium (II) halide catalyst; and (b) subjecting the homogeneous aqueous solution to conditions suitable to produce a product stream comprising acetic acid and hydrogen.

### CROSS REFERENCE TO RELATED APPLICATIONS

This application claims the benefit of priority of U.S. Provisional Patent Application No. 62/343,396 filed May 31, 2016, which is hereby incorporated by reference in its entirety.

### BACKGROUND OF THE INVENTION

### A. Field of the Invention

The invention generally concerns the production of acetic acid and hydrogen from a two carbon (C₂) alcohol source using an organoruthenium (II) halide catalyst.

### B. Description of Related Art

Acetic acid and hydrogen are important industrial chemicals used for the production a various organic compounds. Hydrogen is also an important source of energy. Acetic acid can be used produce commercial products such as vinyl acetate, polyethylene terephthalate (PET), acetic anhydride, and acetate esters. Acetic acid can be produced from methanol carbonylation using methanol and carbon monoxide as shown in equation (1)-(3):

CH₃OH+HI→CH₃I+H₂O (1)

CH₃I+CO→CH₃COI (2)

CH₃COI+H₂O→CH₃COOH+HI (3)

Methanol and carbon monoxide can be sourced from various resources including natural gas, coal, nuclear energy, and other renewable energy sources, such as biomass, wind, solar, geothermal, and hydroelectric power. Both the rhodium-catalyzed Monsanto process and iridium-catalyzed Cativa™ process (BP America, U.S.A.), have been employed for methanol carbonylation. Other processes to produce acetic acid include acetaldehyde oxidation, ethylene oxidation, oxidative fermentation, and anaerobic fermentation.

Commerically, hydrogen is produced from steam reforming of methane as shown in the equations (4) and (5) below. The major source of the methane is from natural gas.

CH₄+H₂O→CO+3H₂ (4)

CO+H₂O→CO₂+H₂ (5)

Alternative processes for hydrogen production have been proposed (for example, water-splitting, thermal dehydrogenation of formic acid, catalytic dehydrogenation of small organic molecules, thermal dehydrogenation of amino-boranes and the like). All of these process suffer from a range of deficiencies, such as high cost and inefficiency, low starting material hydrogen content, and unstable catalysts under reaction conditions requiring high reaction temperatures and pressures. Hydrogen storage and transportation are also problems associated with renewable forms of hydrogen production.

Various attempts to produce acetic acid and/or hydrogen have been disclosed. By way of example Junge et al. in “Novel improved ruthenium catalysts for the generation of hydrogen from alcohols,” Chemical Communications, 2007, 5:522-524 discloses ruthenium complexes useful for the catalysis of alcohol-based feedstocks to produce hydrogen gas and acetaldehyde. Other recent disclosures have shown other catalyst systems, such as mixed copper precipitates for the production of hydrogen and acetic acid from aqueous ethanol. Brei et al, describes the synthesis of acetic acid and hydrogen from aqueous ethanol with supported Cu/ZnO, Cu/ZrO₂, Cu/Al₂O₃, and Cu/ZnO—ZrO₂—Al₂O₃ catalysts (See, for example, “Synthesis of acetic acid from ethanol-water mixture over Cu/ZnO—ZrO₂—Al₂O₃ catalyst,” Applied Catalysis, A: General, 2013, 458:196-200(, Sharanda et al., “Synthesis of acetic acid from a water-ethanol mixture over a Cu/ZnO—ZrO₂—Al₂O₃ catalyst”, Dopovidi Natsional'noi Akademii Nauk Ukraini, 2010, 10:138-142, and Ukrainian Patent No. UA45526). All of the above mentioned mixed copper precipitates catalysts require high reaction temperatures (>250° C.) to obtain any useful conversions of acetic acid and hydrogen.

In view of above and problems associated with alternative means of producing both acetic acid and hydrogen, new economical routes for acetic acid production to meet growing global demands are needed.

### SUMMARY OF THE INVENTION

A discovery has been made that provides a solution to the aforementioned problems and inefficiencies associated with the generation of acetic acid and hydrogen from a two carbon (C₂) alcohol source. The discovery is premised on the use of an organoruthenium (II) halide catalyst under homogeneous aqueous conditions, and provides an elegant way to produce both acetic acid and hydrogen of high purity under robust reaction conditions at ambient temperatures (20° C. to 35° C.) up to 100° C. (20° C. to 100° C., 50° C. to 80° C., or 65 to 75° C.) in a single reaction step as shown below.

C₂ alcohol source→H₂+CH₃CO₂H (6)

The reaction proceeds under aqueous conditions to produce acetic acid and hydrogen from a two carbon (C₂) alcohol source, such as ethanol, acetaldehyde, or hydrated acetaldehyde. The system is oxygen-resilient, chemically robust, and energy efficient, thereby allowing for large scale acetic acid and hydrogen production to meet the ever increasing demands of the chemical and petrochemical industries. Without wishing to be bound by theory, it is believed that the high efficiency of this system is most likely a result of hydrogen evolution occurring in the homogeneous phase. As illustrated in non-limiting embodiments in the examples, cumulative injection of acetaldehyde provides continuous acetic acid and hydrogen production. The current process provides an elegant and aqueous homogenous catalyst system that produces acetic acid and hydrogen of high purity under robust reaction conditions at ambient or near ambient temperatures.

In one particular aspect of the present invention, there is disclosed a method of producing acetic acid and hydrogen from a two carbon (C₂) alcohol source, the method can include obtaining a homogeneous aqueous solution that includes a C₂ alcohol source and an organoruthenium (II) halide catalyst, and subjecting the homogeneous aqueous solution to conditions suitable to produce a product stream that includes acetic acid and hydrogen. The C₂ alcohol source can be ethanol, hydrated acetaldehyde, or a mixture thereof. In one embodiment, the C₂ alcohol source is ethanol. In another embodiment, the C₂ alcohol source is hydrated acetaldehyde. In yet another embodiment, the hydrated acetaldehyde source is acetaldehyde. The homogeneous ruthenium catalyst can be an organoruthenium (II) halide catalyst that includes an aromatic compound, a phenyl group or a substituted phenyl group. In some aspects, the organoruthenium (II) halide catalyst is benzeneruthenium(II) chloride, or dichloro(p-cymene)ruthenium, or a mixture thereof. In one embodiment, the organoruthenium (II) halide catalyst is benzeneruthenium(II) chloride and, in another embodiment, the organoruthenium (II) halide catalyst is dichloro(p-cymene)ruthenium.

In another particular aspect of the current invention, the reaction conditions of the method can include a temperature of 20° C. to 100° C., or 50° C. to 80° C., or 65 to 75° C. and the aqueous solution can further contain a solvent. Preferably, when the method further contains solvent, the solvent has a boiling point greater than 70° C. In some aspects, the solvent is acetonitrile, dimethylformamide, dimethoxyethane, pyridine or mixtures thereof. Another feature of the current method includes incrementally adding additional amounts of the C₂ alcohol source to the aqueous homogeneous solution. The catalyst can have a turnover rate of 20 to 50, 25 to 40, or 28 and the molar ratio of C₂ alcohol source to organoruthenium (II) halide catalyst can be 40 to 1500. In some aspects of the method, the aqueous solution can include potable water, purified water, tap water, or mixtures thereof. In yet another aspect, there is disclosed a composition that can include a homogeneous aqueous solution containing a C₂ alcohol source, an organoruthenium (II) halide catalyst, acetic acid, and hydrogen.

Also disclosed is a system for using the disclosed method of the current invention to produce acetic acid and hydrogen from a C₂ alcohol source. The system can include a reaction zone containing a homogeneous aqueous solution having a C₂ alcohol source and an organoruthenium (II) halide catalyst. A first outlet can be in fluid communication with the reaction zone, and be configured to receive a first portion of the product stream that includes hydrogen gas. A second outlet can be in fluid communication with the reaction zone and be configured to receive a second portion of the product stream that includes acetic acid. In some aspects, the system can also include a separation zone in fluid communication with the second outlet. The separation zone can be configured to separate acetic acid from the second portion of the product stream.

The following includes definitions of various terms and phrases used throughout this specification.

The term “homogeneous”, as used herein, means the catalyst is soluble (i.e., same phase as the reactants) in the reaction solution. In direct contrast, the term “heterogeneous” refers to the form of catalysis where the phase of the catalyst differs from that of the reactants.

The term “aromatic compound” is intended to mean a compound that includes at least one unsaturated cyclic group having delocalized pi electrons. The term is intended to encompass both hydrocarbon aromatic compounds and heteroaromatic compounds. The terms “hydrocarbon aromatic ring” or “hydrocarbon aromatic compound” refer to an aromatic ring or compound in which the aromatic moieties have only carbon and hydrogen atoms. The terms “heteroaromatic ring” or “heteroaromatic compound” refer to an aromatic ring or compound wherein in at least one aromatic moiety one or more of the carbon atoms within the cyclic group has been replaced by another atom, such as nitrogen, oxygen, sulfur, or the like. The definition also includes cyclopentadienyl anions and derivatives since they also satisfy Huckel's rule of 4n+2π-electrons in a planar, cyclic, conjugated molecule.

“Substituted phenyl group” refers to a phenyl or an aryl moiety substituted by at least one substituent that can include a halogen (chlorine, bromine, fluorine or iodine), an amino, a nitro, a hydroxy, an alkyl, an alkoxy, a haloalkyl, a haloalkoxy, a carboxylic acid, an ester, an amide, a nitrile, an acyl, a thiol, a thiolether substituent, and the like.

“Alkyl refers to a straight or a branched chain substituted or unsubstituted, saturated hydrocarbon having 1 to 20 carbon atoms, and includes, for example, methyl, ethyl, propyl, isopropyl, butyl, isobutyl, secondary butyl, tertiary butyl, pentyl, isopentyl, neopentyl, hexyl, heptyl, octyl, 2-ethylhexyl, 1,1,3,3-tetramethylbutyl, nonyl, decyl, dodecyl, tetradecyl, hexadecyl, octadecyl, and eicosyl. Alkyl substituents can include a halogen an amino, a nitro, a hydroxy, an alkyl, an alkoxy, a haloalkyl, a haloalkoxy, a carboxylic acid, an ester, an amide, a nitrile, an acyl, a thiol, a thiolether substituent, and the like.

“Alkoxy” refers to a straight or a branched chain substituted or unsubstituted, saturated hydrocarbon having 1 to 10 carbon atoms, and includes, for example, methoxy, ethoxy, propoxy, isopropoxy, butoxy, isobutoxy, secondary butoxy, tertiary butoxy, pentyloxy, isopentyloxy, hexyloxy, heptyloxy, octyloxy, nonyloxy and decyloxy. Alkoxy substituents can include a halogen an amino, a nitro, a hydroxy, an alkyl, an alkoxy, a haloalkyl, a haloalkoxy, a carboxylic acid, an ester, an amide, a nitrile, an acyl, a thiol, a thiolether substituent, and the like.

“Haloalkyl” refers to straight chain or branched alkyl substituents having 1 to 8 carbon atoms which is substituted by at least one halogen, and includes, for example, chloromethyl, bromomethyl, fluoromethyl, iodomethyl, 2-chloroethyl, 2-bromoethyl, 2-fluoroethyl, 3-chloropropyl, 3-bromopropyl, 3-fluoropropyl, 4-chlorobutyl, 4-fluorobutyl, dichloromethyl, dibromomethyl, difluoromethyl, diiodomethyl, 2,2-dichloroethyl, 2,2-dibromoethyl, 2,2-difluoroethyl, 3,3-dichloropropyl, 3,3-difluoropropyl, 4,4-dichlorobutyl, 4,4-difluorobutyl, trichloromethyl, trifluoromethyl, 2,2,2-trifluoroethyl, 2,3,3-trifluoropropyl, 1,1,2,2-tetrafluoroethyl and 2,2,3,3-tetrafluoropropyl.

“Turn over number” or TON,” refers to the number of moles of substrate that a mole of catalyst converts in the timeframe of the experiment or before being deactivated. TON is calculated as the number of moles of C₂ alcohol source, divided by the number of moles of catalyst unless otherwise indicated.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods and catalysts of the present invention are their abilities to produce acetic acid and hydrogen from an aqueous C₂ alcohol solution.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 shows a schematic of a system of the present invention that includes the homogenous organoruthenium (II) halide catalyst capable producing acetic acid and hydrogen.

FIG. 2 shows a GC-MS chromatograph and mass spectrum of the reaction mixture showing acetaldehyde.

FIG. 3A shows a GC-MS chromatograph and mass spectrum of the reaction mixture showing ethanol.

FIG. 3B shows a GC-MS chromatograph and mass spectrum of the reaction mixture showing acetic acid.

FIG. 4 shows a graphical representation of the reaction output of single injection versus cumulative injection in one embodiment of the current invention.

### DETAILED DESCRIPTION OF THE INVENTION

A discovery has been made that provides a solution to the aforementioned problems and inefficiencies associated with the generation of acetic acid and hydrogen from a two carbon (C₂) alcohol source. The discovery is based, in part, on the reaction of organoruthenium (II) halide catalyst with ethanol, acetaldehyde, or hydrated acetaldehyde under homogeneous aqueous conditions.

These and other non-limiting aspects of the present invention are discussed in further detail in the following sections.

### A. Organoruthenium Catalyst

In one embodiment, the transition metal catalyst of the present invention is a ruthenium halide compound. Non-limiting examples of ruthenium halide compounds include RuCl(p-cymene)[(R,R)-TsDPEN], RuCl(p-cymene)[(S,S)-TsDPEN], RuCl(p-cymene)[(R,R)-FsDPEN], RuCl(p-cymene)[(S,S)-FsDPEN], RuCl(mesitylene) [(R,R) -TsDP EN], RuCl(mesitylene)[(S,S)-TsDPEN], RuCl(mesitylene)[(R,R)-FsDPEN], RuCl(mesitylene)[(S,S)-FsDPEN], [(R,R)-Teth-TsDPEN RuCl], and [(S,S)-Teth-TsDPEN RuCl], dichloro(p-cymene)triphenylphosphineruthenium(II) [RuCl₂(p-cymene)(PPh₃)], dichloro(p-cymene)tricyclohexylphosphineruthenium(II) [RuCl₂(p-cymene)(PCy₃)], cyclopentadienyl(η⁶-napthalene)ruthenium(II) hexafluorophosphate [CpRu(η⁶-napthalene)]⁺ PF₆⁻, cyclopentadienyl(p-cymene)ruthenium(II) hexafluorophosphate [CpRu(p-cymene)]⁺ PF₆⁻, benzeneruthenium(II) chloride dimer, [RuCl₂(benzene)] ₂, benzeneruthenium(II) bromidedimer [RuBr₂(benzene)]₂, benzeneruthenium (II) iodide dimer [RuI₂(benzene)]₂, dichloro(toluene)ruthenium(II) dimer [RuCl₂(toluene)]₂, dibromo(toluene)ruthenium(II) dimer [RuBr₂(toluene)]₂, diido(toluene)ruthenium(II) dimer [RuI₂(toluene)]₂, dichloro(xylene)ruthenium(II) dimer [RuCl₂(xylene)]₂, dibromo(xylene)ruthenium(II) dimer [RuB r₂ xylene)]₂, diido(xylene)ruthenium (II) dimer [RuI₂(xylene)]₂, dichloro(p-cymene)ruthenium(II) dimer [RuCl₂(p-cymene)]₂, dibromo(p-cymene)ruthenium(II) dimer [RuBr₂(p-cymene)]₂, diido(p-cymene)ruthenium(II) dimer [RuI₂(p-cymene)]₂, dichloro(mesitylene)ruthenium(II) dimer [Ru(mesitylene)Cl₂]₂, dibromo(mesitylene)ruthenium(II) dimer [Ru(mesitylene)Br₂]₂, diiodo(mesitylene)ruthenium(II) dimer [Ru(mesitylene)I₂]₂, dichloro(hexamethylbenzene)ruthenium(II) dimer [(C₆Me₆)RuCl₂]₂, dibromo(hexamethylbenzene)ruthenium (II) dimer [(C₆Me₆)RuI₂]₂, diiodo(hexamethylbenzene)ruthenium(II) dimer [(C₆Me₆)RuCl₂]₂, pentamethylcyclopentadienylbis(triphenylphosphine)ruthenium(II) chloride [(C₅Me₅)Ru(PPh₃)₂Cl], chloro(pentamethylcyclopentadienyl)(cyclooctadiene)ruthenium(II) [(C₅Me₅)Ru(COD)Cl], dichloro(pentamethylcyclopentadienyl)ruthenium(III) polymer [(C₅Me₅)RuCl₂], and chloro(μ-methanethioato)(pentamethylcyclopentadienyl)ruthenium(III) dimer [(C₅Me₅)Ru(SMe)Cl]₂. Preferably the ruthenium compound of the present invention is an organoruthenium halide catalyst containing ruthenium metal with an oxidation state of 2⁺ (II) and aromatic compound, such as a phenyl group or a substituted phenyl group having the general structure (I) shown below:

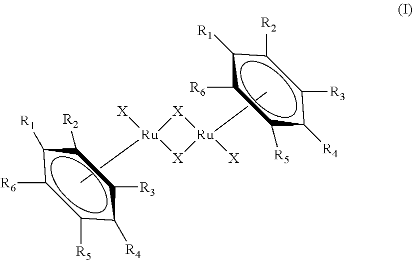

where X can be a halogen (Cl, Br, or I), preferably Cl, and R₁-R₆ can be substituents that are the same or different. R₁-R₆ can be H, C₁ to C₄ alkyl, alkoxy, or haloalkyl moieties, or a combination thereof. In a preferred embodiment, R₁-R₆ are H, R₁-R₅ are H and R₆ is methyl, R₂, R₄, R₅, and R₆ are H and R₁ and R₃ are methyl, R₂, R₄, R₆ are H and R₁, R₃, R₅ are methyl, R₂, R₃, R₅, and R₆ are H and R₁ is methyl and R₄ is isopropyl, or R₁-R₆ are methyl. In a particular embodiment, the organoruthenium (II) halide catalyst is [RuCl₂(benzene)]₂, [RuCl₂(toluene)]₂, [RuCl₂(xylene)]₂, [RuCl₂(p-cymene)]₂, or [RuCl₂(mesitylene)]₂. Non-limiting commercial sources of [RuCl₂(benzene)]₂ and [RuCl₂(p-cymene)]₂ include Sigma-Aldrich®, USA.

### B. Reactants and Medium for Production of Acetic Acid and Hydrogen

1. Reactants

The reactants for producing acetic acid and hydrogen can include a two carbon (C₂) alcohol source, such as ethanol or acetaldehyde. Ethanol can be absolute ethanol, azeotropic distilled ethanol (95%), or aqueous ethanol (for example 50% in water). Acetaldehyde can be anhydrous acetaldehyde, aqueous acetaldehyde solutions (for example 40% in water), paraldehyde (2,4,6-trimethyl-1,3,5-trioxane), metaladehyde (2,4,6,8-tetramethyl-1,3,5,7-tetraoxocanemetacetaldehyde), or combinations thereof. Paraldehyde is the cyclic trimer of acetaldehyde and metaladehyde is the cyclic tetramer of acetaldehyde. Hydrated acetaldehyde can be in acetal form where water and acetaldehyde combine to form equilibrium concentrations of ethane-1,1-diol. Acetaldehyde can also be acetaldehyde in alcoholic solution (for example 50% in ethanol). Acetaldehyde and ethanol can combine to form equilibrium concentrations of hemiacetal 1-ethoxyethanol. Without being limited by theory, ethane-1,1-diol and/or 1-ethoxyethanol can be present in the reactions of the present invention. Ethanol and acetaldehyde are available from many commercial manufacturers, for example, Sigma Aldrich®, U.S.A. In certain embodiments, the molar ratio of C₂ alcohol source to organoruthenium (II) halide catalyst in the reaction medium is 40 to 1500 and all ratios there between including 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 810, 820, 830, 840, 850, 860, 870, 880, 990, 1000, 1010, 1020, 1030, 1040, 1050, 1060, 1070, 1080, 1090, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, and 1490, preferably from 200 to 800, or 300 to 700, or 400 to 500.

2. Medium

The production of acetic acid and hydrogen from ethanol or acetaldehyde can be performed in any type of medium that can solubilize the catalyst and reagents. In a preferred embodiment, the medium is aqueous containing water. Non-limiting examples of water include de-ionized water, distilled water, softened water, salt water, ocean water, river water, tap water, potable water, purified water, rain water, canal water, city canal water or the like. In a particular embodiment, the water includes potable water, purified water, tap water, or mixtures thereof. Additional solvent(s) that are miscible with water and have a boiling point above 50° C., preferably 70° C., can be added to the aqueous medium. A suitable solvent that may be added to the aqueous medium of the present invention include acetonitrile (ACN), dimethylformamide (DMF), dimethylacetamide (DMA), dimethylsulfoxide (DMSO), 1,4-dioxane, dimethoxyethane (DME), tetrahydrofuran (THF), pyridine, acetone, or mixtures thereof. Preferably, the suitable solvent can be acetonitrile, dimethylformamide, dimethoxyethane, pyridine, or mixtures thereof. The amount of solvent or mixtures of solvents that can be added to the aqueous medium can range from 0 to 50 vol. % and any percentage there between including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49%.

### B. Production of Acetic Acid and Hydrogen

The organoruthenium (II) halide catalyst can catalyze the oxidation of a two carbon (C₂) alcohol source to generate acetic acid and hydrogen under oxygen-resilient, chemically robust, and energy efficient reaction conditions. A method to produce acetic acid and hydrogen from a C₂ alcohol source, such as ethanol or acetaldehyde can include obtaining a homogeneous aqueous solution that includes the C₂ alcohol source and the organoruthenium (II) halide catalyst. The homogeneous aqueous solution can then be subjected to conditions suitable to produce a product stream that includes acetic acid and hydrogen. In some embodiments, the reaction medium includes a temperature of 20° C. to 100° C. and any temperature there between, including 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99° C. Specifically, the reaction medium can include a temperature range of 50° C. to 80° C. or 65 to 75° C. In a preferred embodiment, the organoruthenium (II) halide catalyst is soluble in the reaction medium, thus allowing homogenous catalysis to occur. In some embodiments, the hydrogen gas produced from the reaction can be collected in a cylinder and analyzed by GC-TCD and the resulting reaction mixture containing acetic acid can be analyzed by NMR and GC-MS.

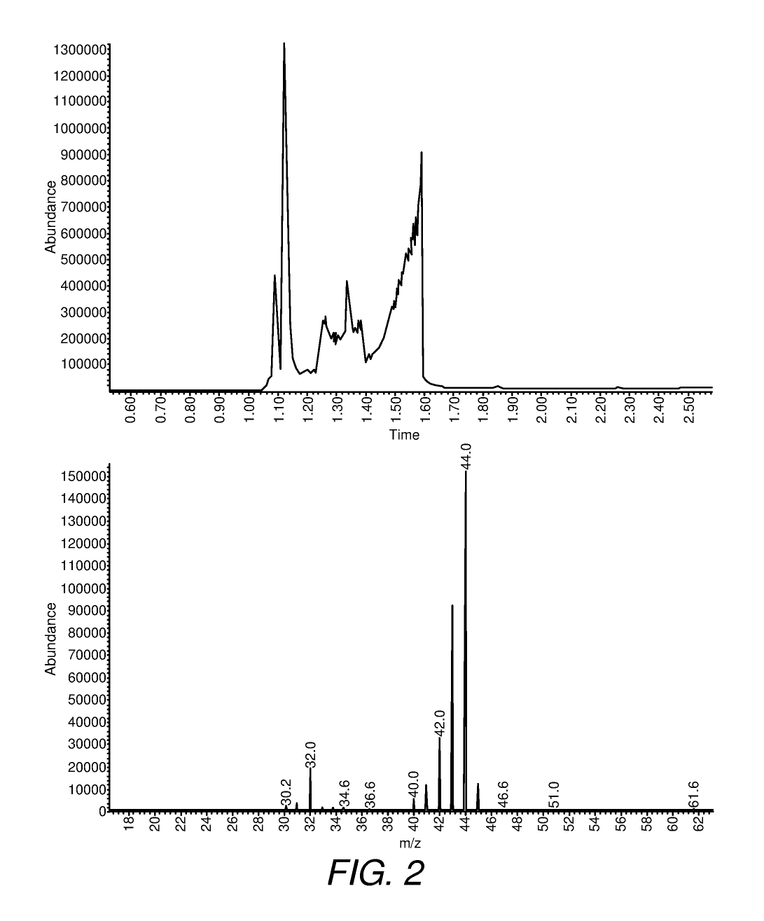

It was surprisingly found that the activity of the catalyst can be increased when incrementally adding additional amounts of the C₂ alcohol source to the aqueous homogeneous medium during the progress of the reaction. Without being limited by theory, it is believed that the catalytic activity can be limited by the concentration of hydrolyzed C₂ alcohol source, which is controlled by C₂ alcohol source/hydrolyzed C₂ alcohol source equilibrium. Lower concentration of C₂ alcohol source can give a lower concentration of hydrolyzed C₂ alcohol source, which can lower activity. In one aspect, the concentration of hydrolyzed C₂ alcohol source can be increased by increasing the concentration of C₂ alcohol source, which can increase catalyst activity. In certain aspects, the C₂ alcohol source is acetaldehyde and the equilibrium is acetaldehyde/ethane-1,1-diol. Additional amounts of the C₂ alcohol source that can be added by cumulative addition to the medium include 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.9, or 1.0 or more equivalents of the C₂ alcohol source added every 10 to 240 minutes or 30 to 120 minutes or more. The addition of additional C₂ alcohol source can be performed using generally known addition techniques (e.g., by injection, dripping, pouring, purging, etc.). In a specific embodiment, the amount of additional C₂ alcohol source added by cumulative injection ranges from 0.4 to 0.5 equivalents added once about every 60 minutes. A non-limiting example of cumulative addition is shown in FIG. 2 of the Examples.

In other embodiments of the method, the catalyst of the present invention can have a turnover rate number (TON) of 20 to 120 or any rate or range there between including 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119 TON. Specifically, in the presence of 0.054 equivalents of C₂ alcohol source the catalyst can have a turnover rate number of 102 after 300 minutes.

### C. System to Produce Acetic Acid and Hydrogen

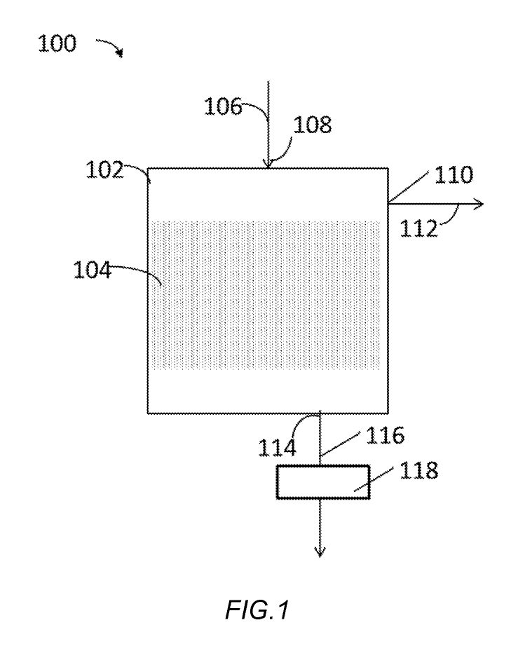

FIG. 1 is a schematic of an embodiment of a system to produce acetic acid and hydrogen. Referring to FIG. 1, system 100 may include a reactor 102 and a reaction zone 104 containing a homogeneous aqueous solution having a C₂ alcohol source and an organoruthenium (II) halide catalyst. In a preferred embodiment, the C₂ alcohol source and the organoruthenium (II) halide catalyst in reaction zone 104 is ethanol, hydrated acetaldehyde, or a mixture thereof, and [RuCl₂(benzene)]₂ or [RuCl₂(p-cymene)]₂ respectively. In some aspects, reactor 102 can optionally be a continuous flow reactor having a reactant stream that includes a feed substrate 106 (C₂ alcohol source) that can enter reactor 102 via an optional feed inlet 108 in one portion, in intermitted portions, or continuously. In preferred embodiments, the system includes a first outlet 110 in fluid communication with reaction zone 104 and the first outlet 104 is configured to receive a first portion of the product stream 112 that includes hydrogen gas. The reactor 102 includes a second outlet 114 in fluid communication with reaction zone 104, which is configured to receive a second portion of the product stream 116 that includes acetic acid. In further embodiments, system 100 may also optionally include a separation zone 118 in fluid communication with the second outlet 114 that is configured to separate acetic acid from the second portion of the product stream. The optional separation zone 118 can be configured with the purpose of preventing the loss of C₂ alcohol source and/or organoruthenium (II) halide catalyst from the homogeneous aqueous solution of reaction zone 104. In some embodiments, the temperature of reaction zone 104 containing a homogeneous aqueous solution having a C₂ alcohol source and an organoruthenium (II) halide catalyst can be operated at a temperature greater than, equal to, or between any two of 20° C., 30° C., 40° C., 50° C., 60° C., 70° C., 80° C., 90° C., 100° C. In a preferred embodiment, the temperature can range from 65 to 75° C. The temperature of reaction zone 104 can be maintained and/or adjusted using generally known heating or cooling techniques.

The resulting acetic acid and hydrogen produced from the systems of the invention can be highly pure. However, if necessary, the resulting acetic acid or hydrogen can be further purified and/or dried using common liquid or gas purification and/or drying techniques, such as vacuum distillation, cryogenic distillation, membrane separation and the like. The system can further include storing the directly produced or subsequently purified and/or dried acetic acid and/or hydrogen gas.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

Catalyst were obtained from Strem Chemicals Inc. U.S.A. Water was deionized under standard conditions. Acetaldehyde (99.5% purity) was obtained from Acros Organics (ThermalFisher Scientific, U.S.A.). Gas chromatograph with athermal conductivity detector (GC-TCD) was performed using an Agilent 7820A GC (Agilent Technologies Inc., U.S.A.) equipped with a TCD and an Agilent HP-Molesieve column. The GC inlet temperature was 45° C. (splitless injection), pressure 3 psi (0.02 MPa) for 2 min, 9 psi/min (0.06 Mpa/min) until end, the TCD had a temperature of 100° C., reference (helium) and ramp rate of 50 mL/min, the GC column temperature was 45° C. for 2.5 min, 20° C. per min till 100° C. and 100° C. for 13 min. NMR was performed on a Bruker AVANCE II 400 (Bruker Corporation, U.S.A.) with Sample Changer. GC-mass spectrometry (GC-MS) was performed using an Agilent 7820A GC equipped with a Agilent 5975 MS detector, with an GC inlet temperature of 200° C. (splitless), a pressure 10 psi (0.068 MPa), a carrier gas (argon) rate of 1.3 mL/min, at 10 psi (0.068 MPa), a GC column temperature of about 40° C. for 1.5 min, 12° C./min till 300° C., 300° C. for 2 min.

### Example 1

### Catalyst Evaluation

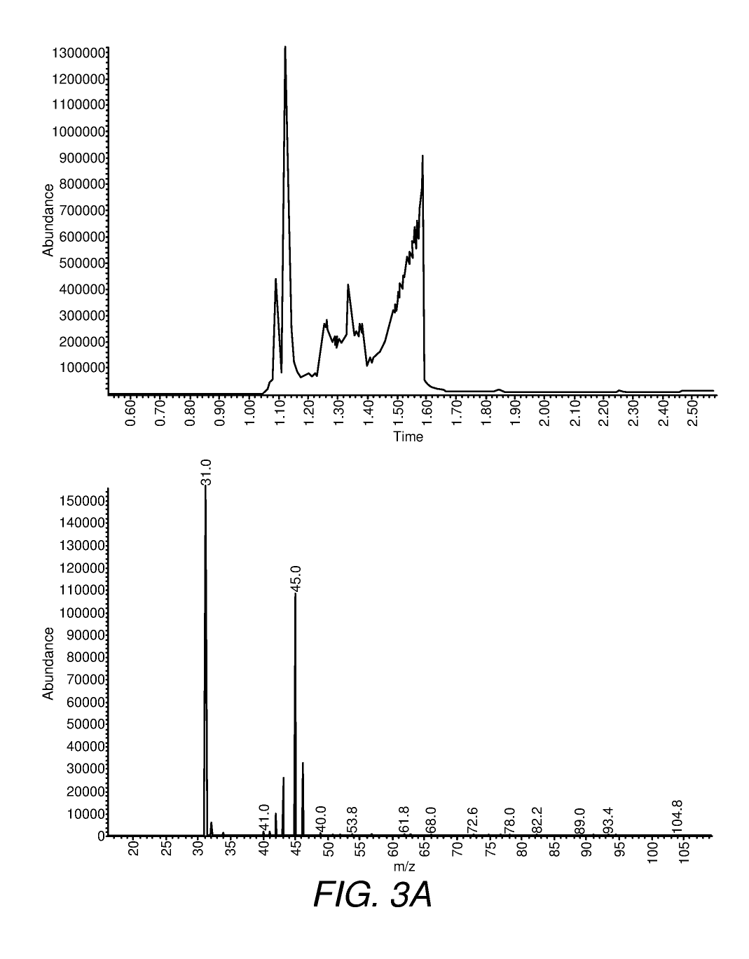

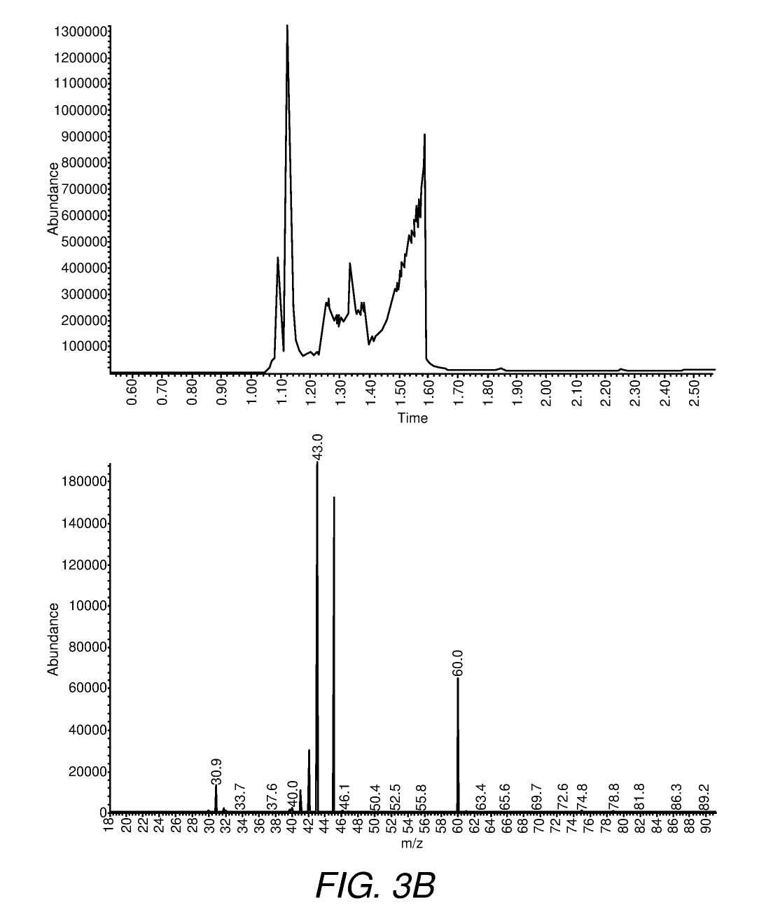

Acetaldehyde (3 mL, 54 mmol) was charged into a reactor fitted with a condenser and diluted with water (50 mL). The catalyst (0.25 mmol) was added and the resultant solution was heated to 70° C. for 3 hours. The amount of acetaldehyde at the start of the reaction was quantified at 43 mmol due to evaporative loss at 70° C. Gas produced from the reaction was collected in a cylinder and analyzed by GC-TCD and the resulting reaction mixture was analyzed by NMR and GC-MS. Mass spectra analysis of the GC peaks at 1.09 min., 1.28 min., and 1.46 to 1.58 min. was performed. Table 1 lists the catalysts that were evaluated and resultant total volume of gas (H₂) produced. As shown from the data in Table 1, only ruthenium catalysts that included an aromatic substituent catalyzed the formation of hydrogen gas and acetic acid from the acetaldehyde. FIGS. 2, 3A, and 3B are GC-MS spectra of the reaction mixture when [Ru(p-cymene)Cl₂]₂ was evaluated. FIG. 2 shows the GC-MS peak at 1.09 min in the reaction mixture, which corresponds to acetaldehyde (m/z of 44). FIGS. 3A and 3B show the GC-MS the peaks 1.28 min and 1.4 to 1.58 min of the reaction mixture, which correspond ethanol (m/z of 45) and acetic acid (m/z of 60) respectively. From the GC-MS and the evolution of hydrogen it was determined that acetic acid was formed from acetaldehyde when [Ru(p-cymene)Cl₂]₂ was used as the catalyst. The formation of ethanol was due to the reduction of acetaldehyde and/or acetic acid.

TABLE 1

| Catalyst | Total gas volume (mL) |
|---|---|
| Blank (no catalyst) | 0 |
| Ru₃(CO)₁₂ | 0 |
| [Ru(p-cymene)Cl₂]₂ | 320 |
| RuCl₂(PPh₃)₃ | 0 |
| [Ru(benzene)Cl₂]₂ | 140 |
| RuCl₃ | 0 |
| Ru(OH)₃/Fe_{nanoparticles} | 0 |
| Ru nitrosylnitrate | 0 |
| Ru(acac)₃ | 0 |
| RuO₂ | 0 |
| IrCl₃ | 0 |

### Example 2

### Double Concentration of Substrate

Acetaldehyde (6 mL, 108 mmol) was charged into a reactor fitted with a condenser and diluted with water (50 mL). [Ru(p-cymene)Cl₂]₂ (150 mg, 0.25 mmol) was added and the resultant solution was heated to 70° C. The amount of acetaldehyde at the start of reaction was 86 mmol due to evaporative loss at 70° C. Gas produced from the reaction was collected in a cylinder and analyzed by GC-TCD and the resulting reaction mixture was analyzed by NMR and GC-MS. The amount of gas produced was 560 mL (H₂, 25 mmol) after 3 h and 930 mL (H₂, 42 mmol) after 22 h.

### Example 3

### Double Injection of Substrate

Acetaldehyde (3 mL, 54 mmol) was charged into a reactor fitted with a condenser and diluted with water (50 mL). [Ru(p-cymene)Cl₂]₂ (150 mg, 0.25 mmol) was added and the resultant solution was heated to 70° C. The amount of acetaldehyde at the start of reaction was 43 mmol due to evaporative loss at 70° C. Gas produced from the reaction was collected in a cylinder and analyzed by GC-TCD, and the resulting reaction mixture was analyzed by NMR and GC-MS. The amount of gas produced was 650 mL (H₂, 28 mmol) after 20 h. After 20 h, no further gas was produced. A second aliquot of acetaldehyde (3 mL, 54 mmol) was injected. The amount of total gas produced after 24 more hours was 1290 mL. Hence, 640 ml (H₂, 28 mmol) of gas was produced for the second aliquot of acetaldehyde.

### Example 4

### Solvent Evaluation

Solvents boiling at greater than 70° C. and miscible with water were evaluated. Acetaldehyde (1 mL, 18 mmol) was charged into a reactor fitted with a condenser and diluted with water or a solvent/water mixture. [Ru(p-cymene)Cl₂]₂ (50 mg, 0.08 mmol) was added and the resultant solution was heated to 70° C. for 1 h. Gas produced from the reaction was collected in a cylinder and analyzed by GC-TCD. Table 2 shows the resultant total volume of gas (H₂) produced and the water: solvent vol. ratio.

TABLE 2

| Solvent | Total gas volume (mL) |
|---|---|
| Water | 70 |
| Water:acetonitrile (1:1) | 32 |
| Water:dimethylformamide (1:1) | 66 |
| Water:dimethoxyethane (1:1) | 52 |
| Water:pyridine (1:1) | 12 |

### Example 5

### Cumulative Addition

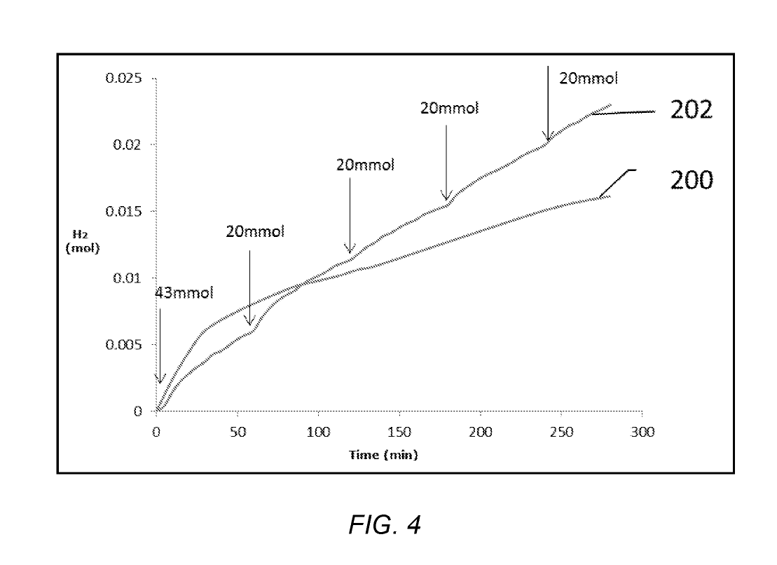

The following reaction was prepared in duplicate and run in parallel. Acetaldehyde (3 mL, 54 mmol) was charged into a reactor fitted with a condenser and diluted with water (50 mL). The amount of acetaldehyde at the start of reaction was 43 mmol due to evaporative loss at 70° C. [Ru(p-cymene)Cl₂]₂ (150 mg, 0.025 mmol) was added to both reactions and the resultant solutions were heated to 70° C. FIG. 4 shows the reaction progress of the two parallel reactions. The first reaction 200 was allowed to progress unchanged but the second reaction 202 was injected with acetaldehyde (1.1 mL, 20 mmol) every hour in cumulative fashion. Gas produced from the reaction was collected in a cylinder and analyzed by GC-TCD and the resulting reaction mixture was analyzed by NMR and GC-MS.

1. A method of producing acetic acid and hydrogen from a two carbon (C₂) alcohol source, the method comprising:
(a) obtaining a homogeneous aqueous solution comprising water, a solvent having a boiling point great than 70° C., a C₂ alcohol source and a organoruthenium (II) halide dimer catalyst; and
(b) reacting the homogeneous aqueous solution at a temperature of 30° C. to 100° C. to produce a product stream comprising acetic acid and hydrogen.

2. The method of claim 1, wherein the C₂ alcohol source is ethanol, hydrated acetaldehyde, or a mixture thereof.

3. The method of claim 2, wherein the C₂ alcohol source is ethanol.

4. The method of claim 2, wherein the C₂ alcohol source is hydrated acetaldehyde.

5. The method of claim 2, wherein the hydrated acetaldehyde source is acetaldehyde.

6. The method of claim 1, wherein the organoruthenium (II) halide catalyst comprises an aromatic compound, a phenyl group or a substituted phenyl group.

7. The method of claim 6, wherein the organoruthenium (II) halide catalyst is benzeneruthenium(II) chloride dimer, or dichloro(p-cymene)ruthenium dimer, or a mixture thereof.

8. The method of claim 7, wherein the organoruthenium (II) halide dimer catalyst is benzeneruthenium(II) chloride dimer.

9. The method of claim 7, wherein the organoruthenium (II) halide dimer catalyst is dichloro(p-cymene)ruthenium dimer.

10. The method claim 1, wherein the reaction temperature is 50° C. to 80° C., or 65 to 75° C.

11. (canceled)

12. The method of claim 1, wherein the solvent is acetonitrile, dimethylformamide, dimethoxyethane, pyridine or mixtures thereof.

13. The method of claim 1, wherein the solvent is acetonitrile.

14. The method of claim 1, further comprising incrementally adding additional amounts of the C₂ alcohol source to the aqueous homogeneous solution.

15. The method of claim 1, wherein the catalyst has a turnover rate of 20 to 50, 25 to 40, or 28.

16. The method of claim 1, wherein the molar ratio of C₂ alcohol source to organoruthenium (II) halide catalyst is 40 to 1500.

17. The method of claim 1, wherein the aqueous solution comprises potable water, purified water, tap water, or mixtures thereof.

18. A composition comprising a homogeneous aqueous solution comprising a C₂ alcohol source, an organoruthenium (II) halide catalyst, acetic acid, and hydrogen.

19-20. (canceled)
